(19) United States
(12) Patent Application Publication
(10) Pub. No.: US 2016/0374934 A1
(43) Pub. Date: Dec. 29, 2016

(54) METHODS FOR TREATMENT OF HAIR GRAYING

(71) Applicant: STEMTECH INTERNATIONAL, INC., Pembroke Pines, FL (US)

(72) Inventors: Christian Drapeau, Austin, TX (US); Gitte S. Jensen, Port Dover (CA)

(73) Assignee: STEMTECH INTERNATIONAL, INC., Pembroke Pines, FL (US)

(21) Appl. No.: 15/039,005

(22) PCT Filed: Nov. 26, 2014
(86) PCT No.: PCT/US14/67734

§ 371 Date: May 24, 2016

Related U.S. Application Data
(60) Provisional application No. 61/909,141, filed on Nov. 26, 2013.

(51) Int. Cl. A61K 8/97; A61Q 5/10; A61K 8/73

(52) U.S. Cl. CPC A61K 8/97; A61K 8/73; A61Q 5/10; A61K 2800/92

(57) ABSTRACT

The invention relates to methods for treating hair graying. More specifically, the present invention discloses new methods for reducing, stopping and/or reversing of hair graying in mammals by stimulating stem cell migration to produce or stimulate melanocytes in hair follicles. The invention further teaches a stem cell mobilizing composition for treating graying hair in a subject, comprising blue-green algae, which stimulates stem cell mobilization and growth of melanocytes in hair follicles.

### FIELD OF THE INVENTION

The present invention relates to methods for treatment of hair graying. More specifically, the present invention relates to methods for reducing, stopping and/or reversing hair graying in mammals by enhancing stem cell.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Changes in hair color that naturally occur as people age is called achromotrichia, and describes the process of colored hair turning gray and then eventually white. Achromotrichia normally begins in the early to mid-twenties in men and late twenties in women, although in some instances white hair can appear as early as childhood. Causative factors for onset of age and extent of hair graying appear to be dominated by genetic factors, although external factors such as stress, medical conditions or other external stimuli almost certainly play a role. While the specific causes of hair graying are unclear, the process by which graying occurs is better understood as occurring largely due to loss of melanin production in the hair root as new hairs grow in without replenishing pigment. It is further understood that stem cells within hair follicles play a central role in the normal regeneration and maintenance of hair. This includes melanin production by melanocyte stem cells—the cells that are primarily responsible for the production and storage of pigment in hair and skin. Once melanocyte stem cell death begins to occurs, the onset of graying begins. Thus, the critical role of stem cells in the hair follicle in aging processes related to hair graying suggests that better understanding of their life cycle and mechanisms for growth and differentiation will provide opportunities to target hair graying processes for reduction, stoppage and/or reversal.

It is well-understood that regenerative processes in the body generally rely extensively on growth and differentiation of adult stem cells (ASCs), such as the described melanocyte stem cells, to maintain and repair of the tissue in which they are found. While, the general view is that local stem cells are primarily involved in maintenance, such as minor repair of the tissue in which they reside, recent studies have reported that ASCs from one tissue may have the ability to develop into cell types characteristic of other tissues. For example, in some instances, the number of new tissue cells found in healing tissue far exceeds the capacity of local stem cells to duplicate and differentiate, thereby suggesting that stem cells coming from other sites are involved in the process of repair. As such, therapeutic approaches targeting certain cosmetic or medical applications, such as hair graying, may benefit significantly from a broader view of promoting stem cell growth for regeneration and maintenance as opposed to focused approaches concerned primarily with the hair scalp and cells present in the hair follicle niche. Accordingly, there is a great need in the art for broader approaches for the treatment of hair related conditions, such as hair graying.

Described herein is are compositions and methods of administering stem cell mobilizing compounds that help reverse, reduce or eliminate hair graying. The present subject matter describes the ability of stem cell mobilizing compositions to reverse hair graying, as promoting the conversion of melanocytes. The improvements were observed in virtually all subjects over prolonged periods of several months, as shown by quantifying of white hair density using photo-colorimetric assays. The described compositions and methods provide a viable solution for promoting hair darkening, improving cosmetic appearance and providing therapeutic approaches.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts the reduction in the L parameter for subjects applying the inventive method, over a six month period of time, in accordance with an embodiment of the present invention.

FIG. 2 depicts the linear trend in the reduction in the L parameter over time for L* values in accordance with an embodiment of the present invention.

FIG. 3 depicts the linear trend in the reduction in the L parameter over time for the Ratio of L* at month>0 to L* at month 0, in accordance with an embodiment of the present invention.

FIG. 4 is (a) an image taken from the back of the head of one subject prior to and 6 months after daily consumption of a stem cell mobilizing composition, in accordance with an embodiment of the present invention. (b) This series of photographs was taken from the back of the head of one participant at baseline and then after 4, 6 and 8 weeks of consumption of 1 gram of StemEnhance, 3 times a day. Photographs taken from left and right sides of the head show similar darkening of hair color.

FIG. 5 shows that consumption of 1 gram of an extract of Polygonum multiflorum (He Shou Wu) triggered at transient increase (18%) in the number of circulating stem cells while Lycium barbarum (Gou Qi Zi) triggered a transient migration of stem cells out of the blood into tissues, measured as a decrease in the number of circulating stem cells (−24%).

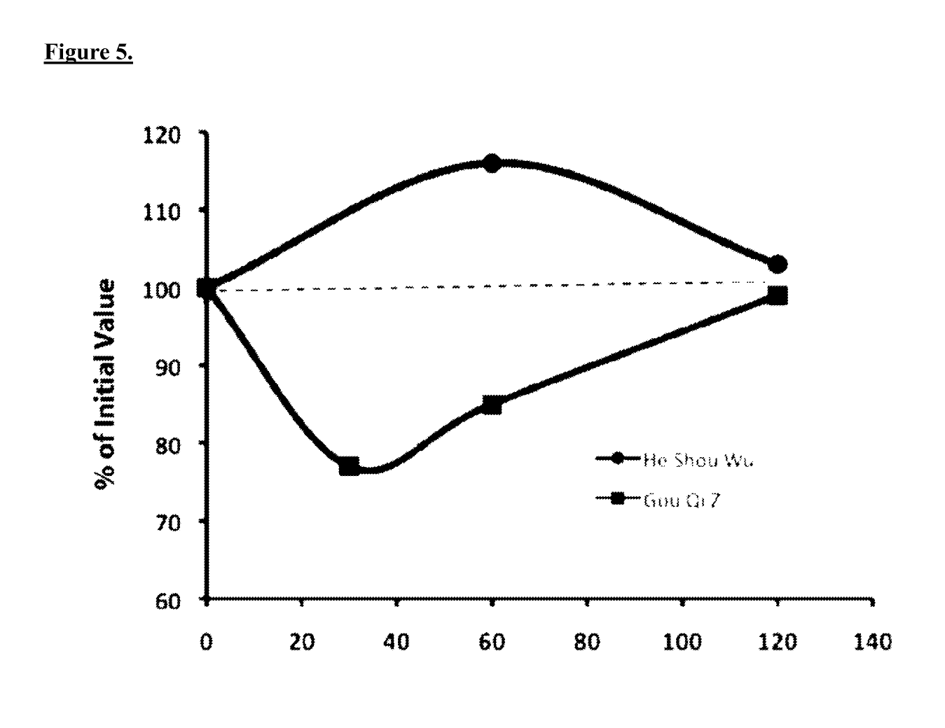

### SUMMARY OF THE INVENTION

Described herein is a method for improving the cosmetic appearance of hair in a subject, including providing a quantity of composition with one or more stem cell mobilization agents selected from the group of: Polygonum multiflorum, blue-green algae, fucoidan, and Lycium barbarum, and a cosmetically acceptable carrier; and administering a cosmetically effective amount of the composition to the subject, wherein administration of the composition improves the cosmetic appearance of hair in a subject. In other embodiments, the stem cell is a bone marrow-derived stem cell (BMSC). In other embodiments, the stem cell is a hematopoietic stem cell (HSC). In other embodiments, administering the quantity includes oral administration. In other embodiments, oral administration includes use of a capsule or a pill. In other embodiments, the capsule or a pill includes 50-750 mg each for the one or more stem cell mobilization agents. In other embodiments, the capsule or a pill includes less than 300 mg of fucoidan and 50-750 mg each for the one or more stem cell mobilization agents selected from the group of: Polygonum multiflorum, blue-green algae, fucoidan, and Lycium barbarum

Described herein is a cosmetic composition including one or more stem cell mobilization agents selected from the group of: Polygonum multiflorum, blue-green algae, fucoidan, and Lycium barbarum, and a cosmetically acceptable carrier. In other embodiments, the composition includes 50-750 mg each for the one or more stem cell mobilization agents in a capsule or pill. In other embodiments, the capsule or a pill includes less than 300 mg of fucoidan and 50-750 mg each for the one or more stem cell mobilization agents selected from the group of: Polygonum multiflorum, blue-green algae, fucoidan, and Lycium barbarum in a capsule or pill. In other embodiments, the composition is for use in treating graying hair in a subject. In other embodiments, the composition is for use in modulating melanocytes in the scalp in a subject.

Further described herein is a method for treating graying hair in a subject, including providing a quantity of composition including one or more stem cell mobilization agents selected from the group of: Polygonum multiflorum, blue-green algae, fucoidan, and Lycium barbarum, and a cosmetically acceptable carrier; and administering a therapeutically effective amount of the composition to the subject, wherein administration of the composition treats hair graying in the subject.

Also described herein is a method of modulating melanocytes in the hair scalp of a subject, including: providing a quantity of composition including one or more stem cell mobilization agents selected from the group of: Polygonum multiflorum, blue-green algae, fucoidan, and Lycium barbarum; and administering the composition to the subject, wherein administration of the composition modulating melanocytes in the hair scape of the subject. In other embodiments, modulating melanocytes in the hair scalp of the subject includes increasing the population of melanocytes in the hair scalp and/or a hair follicle niche. In other embodiments, modulating melanocytes in the hair scalp of the subject includes increasing the production of melanin by melanocytes. In other embodiments, modulating melanocytes in the hair scalp of the subject includes increasing level of melanin keratinocytes. In other embodiments, the composition includes 50-750 mg each for the one or more stem cell mobilization agents in a single dose. In other embodiments, the composition includes less than 300 mg of fucoidan and 50-750 mg each for the one or more stem cell mobilization agents from the group of: Polygonum multiflorum, blue-green algae, fucoidan, and Lycium barbarum

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russell, Molecular Cloning: A Laboratory Manual 3^{th} ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods. For purposes of the present invention, the following terms are defined below.

“Administering” and/or “administer” as used herein refer to any route for delivering a pharmaceutical composition to a patient. Routes of delivery may include non-invasive peroral (through the mouth), topical (skin), transmucosal (nasal, buccal/sublingual, vaginal, ocular and rectal) and inhalation routes, as well as parenteral routes, and other methods known in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

“Blue-green algae” as used herein refers to the gram-negative photosynthetic bacteria belonging to the division Cyanophyta that may exist in unicellular, colonial, or filamentous forms. In addition, the term “blue-green algae” as used herein refers to any fraction, extract, or isolated or purified molecule derived from a blue green algae cell. In one embodiment, the component may be a protein or nucleic acid. In another embodiment, the component may be a phytochemical. In yet another embodiment, the component may be a fraction of a blue green algae. Representative blue-green algae include, but are not limited to, Arthrospira species and Aphanizomenon species. Aphanizomenon flos aquae (“AFA”) is an example of a type of blue-green algae.

“Circulatory system” as used herein refers to the mechanisms for moving blood and blood components throughout the body of a subject, including the vascular and lymph systems. The mechanisms of the circulatory system include, but are not limited to, the heart, blood vessels (arteries, veins, and capillaries), and lymph vessels.

“Component of Lycium barbarum” as used herein refers to any fraction, extract, or isolated or purified molecule from Lycium barbarum. For example, the component is a protein or nucleic acid or a polysaccharide, a phytochemical, or a fraction of Lycium barbarum. Thus, in certain embodiments of the invention, components of Lycium barbarum are obtained by disrupting Lycium barbarum, adding an inorganic or organic solvent, and collecting fractions. Specific, non-limiting examples of fractions are isolated using high performance liquid chromatography, thin layer chromatography, or distillation. Fractionation may be based on the molecular weight or the hydrophobicity of the components of Lycium barbarum.

“Cosmetically effective amount” as used herein refers to the quantity of a specified composition, or active agent in the composition, sufficient to achieve a desired aesthetic effect in a subject being treated. For example, this can be the amount effective for enhancing mobilization and/or migration of stem cells that replenish, repair, or rejuvenate tissue. A cosmetically effective amount may vary depending upon a variety of factors, including but not limited to the physiological condition of the subject (including age, sex, disease type and stage, general physical condition, responsiveness to a given dosage, desired clinical effect) and the route of administration, and can be measured by quantitative improvements in the desired aesthetic trait (e.g., colorimetric density) or subjective satisfaction of the subject or others.

“Differentiation” as used herein refers to the process by which cells become more specialized to perform biological functions. For example, hematopoietic stem cells, hematopoietic progenitors and/or stem cells may change from multipotent stem cells into cells committed to a specific lineage and/or cells having characteristic functions, such as mature somatic cells. Differentiation is a property that is often totally or partially lost by cells that have undergone malignant transformation.

“Enhancement,” “enhance” or “enhancing” as used herein refers to an improvement in the performance of or other physiologically beneficial increase in a particular parameter of a cell or organism, further including cosmetic improvements and aesthetic satisfaction. At times, enhancement of a phenomenon is quantified as a decrease in the measurements of a specific parameter.

“Fucoidan” as used herein describes sulfated fucans obtained from algae. Fucoidan has been obtained from a broad range Algae species as provided in the following non-exhaustive list: Cladosiphon okamuranus, Chordaria flagelliformis, Ch. Gracilis, Saundersella simplex, Desmaestia intermedia, Dictyosiphon foeniculaceus, Dictyota dichotoma, Padina pavonica, Spatoglussum, schroederi, Adernocystis utricularis, Pylayella littoralis, Ascophyllum nodosum, Bifurcaria bifurcata, Fucus. Visculosus, F. spiralis, F. serratus, F. evaescens, Himanthalia lorea, Hizikia fusiforme, Pelvetia canaliculata, P. wrightii, Sargassum stenophyllum, S. honeri, S. Khellmanium, S. muticum, Alaria fistulosa, A. marginata, Arthrothammus bifidus, Chorda film, Ecklonia kurome, E. cava, Eisenia bicyclis, Laminaria angustata, L. brasiliensis, L. cloustoni, L. digitata, L. japonica, L. religiosia, L. saccharina, Macrocystis integrifolia, M. pyrifera, Nereocystis luetkeana, Undaria pinnatifida, Petalonia fascia, Scytosiphon lomentaria. Substantial pharmaceutical research has been done on fucoidan, focusing primarily on two distinct forms: F-fucoidan, which is >95% composed of sulfated esters of fucose, and U-fucoidan, which is approximately 20% glucuronic acid, each of which is included in the term “fucoidan” as used herein. Depending on the source of the fucoidan, fucoidan can serve as a releasing agent in certain embodiments, while in other embodiments, fucoidan can serve as a migration agent.

“Hematopoiesis” as used herein refers to the formation and development of blood cells. Prenatally, hematopoiesis occurs in the yolk sack, then liver, and eventually the bone marrow. In normal adults, it occurs primarily in bone marrow and lymphatic tissues. All blood cells develop from pluripotent stem cells, which are committed to three, two, or one hematopoietic differentiation pathways. This includes the production of hematopoietic cells including B-cells, T-cells, cells of the monocyte macrophage lineage, and red blood cells.

“Hematopoietic agent” as used herein refers to a compound, antibody, nucleic acid molecule, protein, cell or other molecule that affects hematopoiesis. A molecular agent can be a naturally-occurring molecule or a synthetic molecule. In some instances, the agent affects the growth, proliferation, maturation, migration or differentiation or release of hematopoietic cells. In various embodiments, the agent is Lycium barbarum, or an extract or component of Lycium barbarum.

“Hematopoietic stem cells” as used in the present invention means multipotent stem cells that are capable of eventually differentiating into all blood cells including, erythrocytes, leukocytes, megakaryocytes, and platelets. This may involve an intermediate stage of differentiation into progenitor cells or blast cells. The term “hematopoietic progenitors”, “progenitor cells” or “blast cells” are used interchangeably in the present invention and describe maturing HSCs with reduced differentiation potential, but are still capable of maturing into different cells of a specific lineage, such as myeloid or lymphoid lineage. “Hematopoietic progenitors” include erythroid burst forming units, granulocyte, erythroid, macrophage, megakaryocyte colony forming units, granulocyte, erythroid, macrophage, and granulocyte macrophage colony-forming units.

“Homing” as used herein refers to the process of a cell migrating from the circulatory system into a tissue or organ. In some instances, homing is accomplished via tissue-specific adhesion molecules and adhesion processes. Homing may refer to the migration back to the bone marrow.

“Isolated biological component” (such as a nucleic acid molecule, polypeptide, polysaccharide or other biological molecule) as used herein refers to a biological component that has been substantially separated or purified away from other biological components in which the component naturally occurs. Nucleic acids and proteins may be isolated by standard purification methods, recombinant expression in a host cell, or chemically synthesized.

“Lycium barbarum” or “L. barbarum” as used herein refers to a small bright orange-red, ellipsoid berry or fruit grown. One exemplary source is in the north of China, primarily in the Ningxia Hui Autonomous Region. It is sometimes referred to as Gou Qi Zi, goji berry or wolfberry. L. barbarum belongs to the Solanaceae family, the nightshade family that includes hundreds of plant foods like potato, tomato, eggplant, and peppers (paprika).

“Melanocytes” or “melanocyte stem cells” as used herein refers to cells located in the bottom layer (the stratum basale) of the skin's epidermis that produce and store the pigment melanin in the hair and skin. The change in hair color occurs when melanin ceases to be produced in the hair root and new hairs grow without pigment.

“Modulation” or “modulates” or “modulating” as used herein refers to upregulation (i.e., activation or stimulation), down regulation (i.e., inhibition or suppression) of a response or the two in combination or apart.

“Migration” as used herein refers to the central process for movement of cells in the development and maintenance of multicellular organisms. Cells often migrate in response to, and towards, specific external signals, commonly referred to as chemotaxis. Migration includes the process of a cell moving from the circulatory system into a tissue or organ. More specifically, circulating stem cells are tethered to the surface of capillary endothelium via expression of adhesion molecules of cell surfaces, resulting in cytoskeletal changes in both endothelium and stem cells, and allowing movement through the capillary wall en route to a tissue and/or organ site. In some instances, homing is accomplished via tissue-specific adhesion molecules and adhesion processes.

“Migration agent” as used herein are mobilization agents capable of promoting the process of a cell moving from the circulatory system into a tissue or organ. Migration of stem cells may be demonstrated, for example, by a decrease in circulating stem cells in the circulatory or immune system, or by the expression of surface markers and/or adhesion molecules on cell surfaces, which relate to homing, tethering, and/or extravasation of circulating stem cells to the surface of vessels such as capillary endothelium. Examples of migration agents include isolated or purified components extracted from Lycium barbarum, including a polysaccharide-rich fraction (fraction A) of Lycium barbarum extract, colostrum, including a protein-rich fraction (fraction B) of colostrum extract, fucoidan, including an isolated component or compound extracted from an algae, such as a compound found in a polysaccharide-rich fraction (fraction C) of algae extracts, including Chordaria cladosiphon, or other algaes, or extracts thereof, mushrooms, including an isolated component or compound extracted from a mushroom, such as a compound found in a polysaccharide-rich fraction (fraction D) of mushroom extracts, including Cordyceps sinensis or an extract thereof, Ganoderma lucidum or an extract thereof, Hericium erinaceus or an extract thereof, spirulina, including Arthrospira platensis, Arthrospira maxima, or extracts thereof. In different embodiments, this agent affects the migration of stem cells, such as CD34^{high} (CD34+) cells. In one embodiment, the migration agent decreases the number of bone marrow-derived stem cells and/or hematopoietic stem cells circulating in the peripheral blood. In another embodiment, the migration agent relates to enhanced expression of CXCR4 on circulating stem cells.

“Mushroom polysaccharides” as used herein refers to glucans found mainly in various species of mushrooms such as Cordyceps sinesis, Hercicium erinaceous, and Ganoderma lucidum. This also includes the numerous bioactive polysaccharides or polysaccharide-protein complexes from medicinal mushrooms that may enhance innate and cell-mediated immune responses, and exhibit antitumor activities in animals and humans.

“Pharmaceutically acceptable carriers” as used herein refer to conventional pharmaceutically acceptable carriers useful in this invention.

“Polygonum multiflorum” or “P. multiflorum”, as used herein, refers to a species of herbaceous perennial vine growing to 2-4 m tall from a woody tuber native to central and southern China. Leaves are 3-7 cm long and 2-5 cm broad, broad arrowhead-shaped, with an entire margin. Flowers are 6-7 mm diameter, white or greenish-white, produced on short, dense panicles up to 10-20 cm long. Fruit is an achene 2.5-3 mm long. It is also known as Fallopia multiflora, Radix Polygoni, Radix Polygoni Multiflori, fleeceflower, He Shou Wu, or Fo-Ti.

“Polysaccharide” as used herein refers to a polymer of more than about ten monosaccharide residues linked glycosidically in branched or unbranched chains.

“Progenitor cell” as used herein refers to a cell that gives rise to progeny in a defined cell lineage.

“Promote” and/or “promoting” as used herein refer to an augmentation in a particular behavior of a cell or organism. In one embodiment, promoting relates to the mobilization of melanocyte derived stem cells. In another embodiment, promoting relates to the differentiation of stem cells into melanocytes.

“Recruitment” of a stem cell as used herein refers to a process whereby a stem cell in the circulatory system migrates into specific site within a tissue or organ. Recruitment may be facilitated by a compound or molecule, such as a chemoattractant signal or cell receptor. For example, both CXCR4 and SDF-1 have identified roles in stem cell homing and migration.

“Releasing agent” as used herein are mobilization agents capable of promoting the release and egress of stem cells from a tissue of origin. Release of stem cells from a tissue of origin may be demonstrated, for example, by an increase in circulating stem cells in the circulatory or immune system, or by the expression of markers related to egress of stem cells from a tissue of origin, such as bone marrow. Examples of releasing agents include fucoidan, as obtained from an extract of algae such as Undaria pinnatifida. In one embodiment, the releasing agent increases the number of bone marrow-derived stem cells and/or hematopoietic stem cells in the peripheral blood. In another embodiment, the releasing agent affects the number of stem cells, such as CD34^{high} (CD34+) cells, circulating in the peripheral blood.

“Satellite cell” as used herein refers to a muscle-specific stem cell, often located in the periphery of muscle tissue, and capable of migrating into a muscle to aid in tissue repair and reconstruction.

“Stem cells” as used herein are cells that are not terminally differentiated and are therefore able to produce cells of other types. Characteristic of stem cells is the potential to develop into mature cells that have particular shapes and specialized functions, such as heart cells, skin cells, or nerve cells. Stem cells are divided into three types, including totipotent, pluripotent, and multipotent. “Totipotent stem cells” can grow and differentiate into any cell in the body and thus, can form the cells and tissues of an entire organism. “Pluripotent stem cells” are capable of self-renewal and differentiation into more than one cell or tissue type. “Multipotent stem cells” are clonal cells that are capable of self-renewal, as well as differentiation into adult cell or tissue types. Multipotent stem cell differentiation may involve an intermediate stage of differentiation into progenitor cells or blast cells of reduced differentiation potential, but are still capable of maturing into different cells of a specific lineage. The term “stem cells”, as used herein, refers to pluripotent stem cells and multipotent stem cells capable of self-renewal and differentiation. “Bone marrow-derived stem cells” are the most primitive stem cells found in the bone marrow which can reconstitute the hematopoietic system, possess endothelial, mesenchymal, and pluripotent capabilities. Stem cells may reside in the bone marrow, either as an adherent stromal cell type, or as a more differentiated cell that expresses CD34, either on the cell surface or in a manner where the cell is negative for cell surface CD34. “Adult stem cells” are a population of stem cells found in adult organisms with some potential for self-renewal and are capable of differentiation into multiple cell types. Other examples of stem cells are marrow stromal cells, bone mesenchymal stem cells, HSC, multipotent adult progenitor cells (MAPCs), very small embryonic-like stem cells (VSEL), epiblast-like stem cell (ELSC) or blastomere-like stem cell (BLSC).

“Stem cell circulation agent” (SCCA), “mobilization agent”, and/or “mobilization factor” as used herein refers to one or more compounds, antibodies, nucleic acid molecules, proteins, polysaccharides, cells, or other molecules, including, but not limited to, neuropeptides and other signaling molecules, that affects the release, circulation, homing and/or migration of stem cells from the circulatory system into tissue or organ. A molecular agent may be a naturally occurring molecule or a synthetic molecule. Examples of mobilization agents include “releasing agents”, wherein a releasing agent is capable of promoting the egress of stem cells from a tissue of origin and also “migration agents”, wherein a migration agent is capable of promoting the process of a cell moving from the circulatory system into a tissue or organ.

“Subject” as used herein includes all animals, including mammals and other animals, including, but not limited to, companion animals, farm animals and zoo animals. The term “animal” can include any living multi-cellular vertebrate organisms, a category that includes, for example, a mammal, a bird, a simian, a dog, a cat, a horse, a cow, a rodent, and the like. Likewise, the term “mammal” includes both human and non-human mammals.

“Succulent” as used herein refers to all species of plants within the family Agavaceae, Cactaceae, Crassulaceae, Aizoaceae, Apocynaceae, Didiereaceae, Euphorbiaceae, Asphodelaceae, Portulacaceae. This further includes plants known to possess storage organs adapted for water retention, wherein the storage organs are located in the leaf, stems, roots, or any other location.

“Therapeutically effective amount” as used herein refers to the quantity of a specified composition, or active agent in the composition, sufficient to achieve a desired effect in a subject being treated. For example, this can be the amount effective for enhancing migration of stem cells that replenish, repair, or rejuvenate tissue. In another embodiment, a “therapeutically effective amount” is an amount effective for enhancing trafficking of stem cells, such as increasing release of stem cells, as can be demonstrated by elevated levels of circulating stem cells in the bloodstream. In still another embodiment, the “therapeutically effective amount” is an amount effective for enhancing homing and migration of stem cells from the circulatory system to various tissues or organs, as can be demonstrated be decreased level of circulating stem cells in the bloodstream and/or expression of surface markers related to homing and migration. A therapeutically effective amount may vary depending upon a variety of factors, including but not limited to the physiological condition of the subject (including age, sex, disease type and stage, general physical condition, responsiveness to a given dosage, desired clinical effect) and the route of administration. One skilled in the clinical and pharmacological arts will be able to determine a therapeutically effective amount through routine experimentation.

“Trafficking” as used herein refers to the process of movement of a cell from the tissue of origin, traveling within the circulatory or immune system, and localization towards a site within a tissue and/or organ. Trafficking also includes stem cell mobilization, beginning with release from a tissue of origin, such as egress of stem cells from bone marrow. Trafficking further includes movement of a cell from the tissue of origin, homing by adhesion to the endothelium, transmigration, and final migration within the target tissue and/or organ. Furthermore, trafficking may include the process of movement of a cell of the immune system. One specific, non-limiting example of trafficking is the movement of a stem cell to a target organ, also referred to as migration. Another specific, non-limiting example of trafficking is the movement of a B-cell or a pre-B-cell leaving the bone marrow and moving to a target organ.

“Treat,” “treating” and “treatment” as used herein refer to both therapeutic treatment and prophylactic or preventative measures, wherein the object is to prevent or slow down (lessen) the targeted condition, disease or disorder (collectively “ailment”) even if the treatment is ultimately unsuccessful. Those in need of treatment may include those already with the ailment as well as those prone to have the ailment or those in whom the ailment is to be prevented.

Stem cells in the body are recruited to sites of tissue in need of repair and regeneration through processes of mobilization, homing, extravasation, and migration. The mobilization of stem cells and subsequent migration to various tissues sites in the body relies on a combination of mechanical and chemoattractant signals. For example, circulating stem cells in the peripheral blood stream are described in U.S. Pat. Pub. No. 2013/0108587, U.S. App. No. 61/837,045, PCT Pub. No. WO 2012/006100, and WO 2013/074801, which are each fully incorporated by reference. For example, mechanical force or other factors may activate L-selectins on the surface of stem cells, and activation of L-selectins, in turn, may promote elevated expression of the receptor, CXCR4. Cells at the site of tissue injury may also secrete SDF-1 ligand, thereby attracting stem cells expressing receptor CXCR4 to the tissue site. This interaction of SDF-1 and CXCR4 promotes sufficient adhesion to halt circulation of a stem cell in the peripheral blood stream. Based on this model, various dietary supplements can exploit this mechanism to support the phenomenon of natural regeneration and repair in the body. This includes, for examples, L-selectin blockers such fucoidan, that are demonstrated to mobilize HSCs into the bloodstream.

Another stem cell mobilization agent includes the dried root tuber of Polygonum multiflorum plant, also known as fleeceflower root, which has been used as a traditional Chinese medicine called He Shou Wu. Interestingly, as related to hair graying in particular, this medication originally gained notoriety in traditional Chinese medicine from a tale of a famous Chinese military officer condemned to death and jailed without food or drink. Surviving by consuming the leaves and roots of the vinelike weed arising from the floor of his cell, Polygonum multiflorum, the officer's captors later found his remains as still having lustrous black hair. While the origins of this tale are apocryphal, they serve to illustrate the long-held notion that Polygonum multiflorum possesses important properties for tapping into the regenerative and restorative potential of the body.

Interestingly, traditional Chinese medicine texts clearly distinguish a pre-natal Jing and post-natal Jing, with post-natal Jing ends up functioning as Qi, an active principle “life force” forming part of living thing. In the view of traditional Chinese medicine, it is believed that aging results when the Jing is no longer vibrant, and manifests itself in the loss of vitality, color in hair, skin tone, etc. The precious Jing that is stored in the kidneys is considered to be more finite than the other essential substances. Because of this, substances able to nourish or enhance the actual Jing is very highly treasured in Chinese herbal medicine.

Polygonum multiflorum is an example of such an herb which is categorized in traditional Chinese medicine as an herb to tonify the blood, and classical descriptions of the herb attributes includes contribution to an individual's Jing, a finite, physical substance believed to be inherited at conception and stored by the kidneys. As Polygonum multiflorum is primarily categorized as an herb that acts as a tonic for the blood, and because the Jing is regarded as a critical precursor to the production of blood the connection between the two has been somewhat combined. The herb has been reported as helpful to ward off premature aging, premature graying, or loss of hair, dry skin, sore back and knees, fading eyesight, loss of memory, due to its apparent capacity to support and nourish kidney Jing as well as tonify blood.

Remarkably, the view of pre-natal Jing and post-natal Jing in traditional Chinese medicine mirrors modern scientific understanding of regenerative processes in the body, as reliant on embryonic and adult stem cells for generation, regeneration, and repair mechanisms in the body. These traditional viewpoints have been backed up by recent scientific studies have confirming that extracts of Polygonum multiflorum do contain specific molecules related to stem cells growth and differentiation. For example, several molecules contained within Polygonum multiflorum, such as hydroxyl stilbenes, are indeed capable of promoting hair follicle growth, through increased expression of sonic hedgehog (Shh) and β-catenin expression—two important pathways involved in both early embryogenesis and maintaining stem cell identity.

A potentially similar stem cell mobilization agent, StemEnhance, is a concentrate from the cyanophyta Aphanizomenon flos-aquae that enhances the release of adult stem cells from the bone marrow. Having drawn the parallel between what ancient Chinese Medicine referred to as the Jing and adult stem cells discovered by modern science, it is of great interest to compare the effects of StemEnhance on the body to Polygonum multiflorum, or other stem cell mobilization agents. Other examples of stem cell mobilization agents include the previously described sulfated fucan, fucoidan. Fucoidan from one species, Undaria pinnatifida can result in a significant elevation in the number of circulating CD34+ HSCs, with increases of 17%, 23% (P<0.02) and 32% ((P<0.02) occurring at 45, 90 and 180 minute measurement intervals, thereby demonstrating efficacy as a releasing agent. Another example includes the Aloe macroclada species endemic to Madagascar. Oral administration of Aloe macroclada from the Aloe genus for potential to enhance stem cell mobilization in the peripheral bloodstream of human subjects when y measured at 60, 120, 180 and 240 minutes as shown, with a rapid increasing rate of over 60 to 120 minute time points, sustained through subsequent measurements at 180 and 240 minutes.

By contrast, certain other types of mobilization agents also relate to migration of stem cells. In this regard, it is interesting to note that in traditional Chinese medicine, Polygonum multiflorum is rarely used alone when the intent is to support the Jing or the Kidney or to build the blood, instead it is blended with plants that can “circulate” the Jing and the herb, Gou Qi Zi, is commonly used for that purpose. This medicinal herb is also referred to as goji berry or wolfberry, referring to the species Lycium barbarum. Previous studies have shown that Lycium barbarum is capable of triggered a strong transient decrease in circulating stem cells, with a peak decrease in circulating CD34+ cells observed at about 1-2 hours after consumption, and with the number of circulating CD34+ cells was decreased by 30% below the control value, and with observed decrease in the number of circulating stem cells being accompanied by an increase in the expression of CXCR4 on the membrane of circulating stem cells. Remarkably this modern, demonstrated capacity of Lycium barbarum to function as a migration agent, and Polygonum multiflorum as a release agent, mirrors the traditional Chinese medicine concepts wherein distinct processes are involved that ultimately constitute the broader mechanism of stem cell “circulating” from the blood for regeneration and maintenance of the body

As traditional concepts have suggested the possibly pronounced effects of stem cell circulation when provided via combinations of different agents, such as both release agents and migration agents, there is great interest in further confirming such understanding with modern science. And based on the described role of stem cells in hair graying, there is particular interest in exploiting such circulation mechanisms in finding applications for this particular therapeutic and/or cosmetic need.

Although gray hair is the most obvious and universal sign of the aging process, only recently has the mechanism behind hair graying been elucidated. The pigments responsible for hair color are produced by melanocyte stem cells associated with the hair follicles. Apoptosis or loss of melanocyte stem cells is the underlying cause of hair graying. Drugs such as busulfan (given after bone marrow transplants) cause loss of melanocyte stem cells and interfere with melanosome maturation and transfer, which leads to depigmentation of hair color.

There are 2 types of melanin polymers whose combination determines overall hair color. Eumelanin is found in hair and skin, and colors hair grey, black, yellow, and brown. Pheomelanin imparts a pink to red hue and is thus found in particularly large quantities in red hair. Melanocytes differentiate from primitive stem cells residing in the bulge region of the hair follicle. The maintenance of the melanocyte population takes place through the differentiation of hair follicle stem cells into melanocytes. The newly melanocyte stem cells then migrate into the hair matrix as they differentiate into pigment producing melanocytes, where they transfer pigment to colorless keratinocytes responsible for hair. Studies have found that as we age the reservoir of melanocyte stem cells become depleted and the extent of this depletion is proportional to the loss of hair color. In addition, aging is also linked to impaired migration of melanocyte stem cells that end up at locations in the hair follicle where they are unable to contribute to hair color. Lastly, the hair follicle is especially sensitive to oxidation and the oxidative stress that accumulates through the aging process contributes to the selective apoptosis of melanocyte stem cells or simple loss of “stemness”.

Recent studies strongly suggest that stem cells originating from bone marrow compartments significantly contribute to the repair of damaged or degenerating tissues, and that the number of circulating stem cells is a key parameter in the regenerative role of such cells released into peripheral blood. A link to aging processes in the hair scalp and hair follicles is suggested by studies demonstrating stem cells originating from the bone marrow as having the ability of differentiating into hair follicle stem cells with the possibility of forming melanocytes. Importantly, it has been further reported that stem cells from these two stem cell niches in the body have remarkable similarities. For example, when comparing rat follicular dermal cells and bone marrow mesenchymal stem cells from the same animals, adherent hair follicle dermal cells possess fibroblastic morphology in serum-containing culture medium, were CD44(+), CD73(+), CD90(+), and CD34(+). Bone marrow mesenchymal stem cells show a similar morphology and population doubling time and expressed the same cell-surface markers. Both types of cells are known to possess capacity to differentiate into various mesenchymal lineages, such as osteoblasts, adipocytes, chondrocytes, and myocytes and expressed neuroprogenitor cell markers. Telomerase activity in follicle dermal stem cells and bone marrow mesenchymal are also reported to be similar and capable of clonal expansion.

Within this context, it stem cells mobilized by pharmaceutical compositions can migrate into damaged muscles following cardiotoxin injury and to strongly contribute to tissue repair within 5 weeks. In such cases, it is believed that inflammatory cytokines are playing a crucial role in attracting circulating stem cells to the site of injury. In the case of a gradual loss of melanocytes, it is not known whether the affected area can release such cytokines. Nevertheless, the migration of peripheral blood stem cells to the skin and their conversion into melanocytes provides a compelling approach to tackling problems associated with aging of the scalp, degeneration of the hair follicle niche and the resultant hair graying.

Based on these cellular mechanisms, the Inventors hypothesized that daily administration of a stem cell mobilization agent, can support the repopulation of the stem cell reservoir in the bulge region of the hair follicle, leading to increased production of melanin and restoration of hair color.

Described herein are compositions and methods for reducing or reversing the loss of melanocyte stem cells in a hair follicle. The migration of bone marrow stem cells can contribute to the formation of keratinocytes, as well as skin appendages including hair follicles and sweat and sebaceous glands. Hair color is due to pigments produced by melanocytes associated with the hair follicle. In testing conducted with the inventive compositions and methods, application of the composition stimulated circulation of stem cells and migration to hair follicles, which stimulated pigment production and reversed hair graying by up to 28% after 6 months.

The present invention discloses new methods for reducing, stopping and/or reversing the graying of hair in humans by stimulating stem cell migration to produce or stimulate melanocytes in hair follicles. Without being bound by any particular theory, the Inventors believed that administration of the inventive compositions leads to enhanced stem cell mobilization, resulting in the production or stimulation of melanocytes in mammals to reduce, stop and/or reverse hair graying.

Described herein is a cosmetic composition including a stem cell mobilization agent, and a cosmetically acceptable carrier. In various embodiments, the mobilization agent is a composition including one or more of the following components selected from the group of: Aloe or extracts thereof, Polygonum multiflorum or extracts thereof, Lycium barbarum or extracts thereof, colostrum or extracts thereof, spirulina or extracts thereof, fucoidan, Hericium erinaceus or extracts thereof, Ganoderma Lucidum or extracts thereof, and/or Cordyceps Sinensis or extracts thereof. In various embodiments, the stem cell mobilization agent includes Polygonum multiflorum, blue-green algae, fucoidan, Lycium barbarum, analogs thereof, derivatives thereof, extracts thereof, synthetic or pharmaceutical equivalents thereof, fractions thereof, and combinations of any of the foregoing items. The mobilization agents may be combined together in one or more compositions or they may be administered or consumed separately as part of a regimen. They may have individual physiological effects, additive effects and/or synergistic effects with one another, such as serving as both a releasing agent and migration agent. In some embodiments, the mobilization agent is capable of functioning as a migration agent, promoting the process of a cell moving from the circulatory system into a tissue or organ. In some embodiments, the mobilization agent is capable of functioning as a releasing agent, promoting the release and egress of stem cells from a tissue of origin.

In various embodiments the blue-green algae is a gram-negative photosynthetic bacteria belonging to the division Cyanophyta. In various embodiments, the blue-green algae that is a gram-negative photosynthetic bacteria belonging to the division Cyanophyta includes Arthrospira species and Aphanizomenon species. In other embodiments, fucoidan includes Undaria species.

In some embodiments, the subject consumes and digests whole Polygonum multiflorum root, leaves, stem, seeds, fruits, and/or other plant parts. The whole Polygonum multiflorum root, leaves, stem, seeds, fruits, and/or other plant parts may be fresh, frozen, freeze-dried, dehydrated, fermented, or preserved in some other manner. Therefore, Polygonum multiflorum, as described herein, encompasses whole Polygonum multiflorum root, leaves, stem, seeds, fruits, and/or other plant parts. In other embodiments, the mobilization agent is an extract of Polygonum multiflorum, or an isolated component or compound extracted from Polygonum multiflorum, such as a compound found in a polysaccharide-rich fraction of Polygonum multiflorum extracts, or a fraction soluble in aqueous solutions, or a fraction soluble in organic solvents. Polygonum multiflorum can be provided alone as an isolated or purified substance, or may be part of a composition including a pharmaceutically acceptable carrier. In one embodiment, Polygonum multiflorum or extracts thereof is capable of functioning as a migration agent. In one embodiment, Polygonum multiflorum or extracts thereof is capable of functioning as a releasing agent.

Extracts of components found in Polygonum multiflorum include anthraquinones and derivatives, hydroxyl stilbenes, lecithin, chrysophanol, chrysophanic acid, chrysophanol anthrone, emodin, physcion, rhein, chrysophanic acid anthrone, resveratrol, piceid, 2,3,5,4′-tetrahydroxystilbene-2-O—O-D-glucopyranoside, 2,3,5,4′-tetrahydroxystilbene-2-O-β-D-glucopyranoside-2″-O-mo-nogalloyl ester, 2,3,5,4′-tetrahydroxystilbene-2-O-β-D-glucopyranoside-3″-O-monogalloyl ester, 2,3,5,4′-tetrahydroxystilbene-2-O-β-D-glucoside, gallic acid, catechin, epicatechin, 3-O-galloyl(−)-catechin, 3-O-galloyl(−)-epicatechin, 3-O-galloyl-procyanidin B-2,3,3′-di-O-galloyl-procyanidin B-2, and β-sitosterol.

The identity and nature (e.g., stability) of components in prepared Polygonum multiflorum extracts may vary depending on the method used for extraction. For example, water extraction is a leading method of exacting components from Polygonum multiflorum. However, certain components, such as anthraquinones and derivatives are very insoluble in water. Anthraquinones and derivatives are also insoluble in organic solvents at room temperature, but soluble in hot organic solvents (e.g., boiling temperature), such as methanol or ethanol. Similarly, 2,3,5,4′-tetrahydroxystilbene-2-O-β-d-glycoside is known to readily degrade in aqueous solutions in a temperature and pH dependent manner. Therefore, it is understood that extracts of Polygonum multiflorum may be prepared according to any method known in the art. This includes, water extraction, organic solvent extraction (e.g., U.S. patent application Ser. No. 12/006,221), or combinations of such exemplary methods (e.g., admixtures). Examples of organic solvents that may be used include methanol, n-hexane, ethyl acetate, and n-butanol. Combinations of two or more water and/or organic solvents could be added together to generate additional partition layers for extracting different components in different partition layers. Similarly, extracts from Polygonum multiflorum may be prepared from fresh, unprocessed whole plants or parts thereof, or extracts may be prepared from processed Polygonum multiflorum whole plants or parts thereof. For example, processing may be performed by any known method in the art, one example being fermentation. Processing may improve bioavailability of the components in extracts from Polygonum multiflorum, such as through fermentation with bacteria such as Lactobacillus sp. or through addition of black beans.

In some embodiments, the subject consumes and digests whole blue-green algae such as Aphanizomenon flus aquae (AFA). Blue-green algae may be fresh, frozen, freeze-dried, dehydrated, or preserved in some other manner. In one embodiment, the mobilization agent is an extract of blue-green algae, or an isolated component or compound extracted from blue-green algae, such as a compound found in a polysaccharide-rich fraction of blue-green algae extract, or a compound in a water soluble compartment of an blue-green algae extract. Blue-green algae can be provided alone as an isolated or purified substance, or may be part of a composition including a pharmaceutically acceptable carrier. In one embodiment, blue-green algae is capable of functioning as a migration agent. In one embodiment, blue-green algae is capable of functioning as a releasing agent.

In some embodiments, the subject consumes and digests whole spirulina. The spirulina may be fresh, frozen, freeze-dried, dehydrated, or preserved in some other manner. In one embodiment, the mobilization agent is Arthrospira platensis, Arthrospira maxima, or an extract thereof. Spirulina can be provided alone as an isolated or purified substance, or may be part of a composition including a pharmaceutically acceptable carrier. In one embodiment, spirulina is capable of functioning as a migration agent. In one embodiment, spirulina is capable of functioning as a releasing agent.

In some embodiments, the subject consumes and digests whole algae such as Undaria pinnatifida. The algae may be fresh, frozen, freeze-dried, dehydrated, or preserved in some other manner. In alternative embodiments, an extract of the algae is provided or administered to the subject. In another embodiment, the algae encompasses both whole plant and/or extracts thereof. In another embodiment, the algae can be provided alone as an isolated or purified substance, or may be part of a composition including a cosmetically acceptable carrier. In another embodiment, the extract is a highly sulfated, polyanionic soluble fiber. In one embodiment, the extract is an isolated fucoidan. In a different embodiment, the fucoidan is purified following isolation. In an alternative embodiment, a polysaccharide fraction is administered to the subject. In another embodiment, the highly sulfated, polyanionic soluble fiber is administered to the subject. In one, the isolated fucoidan is administered to the subject. In a different embodiment, the purified fucoidan is administered to the subject. In one embodiment, Undaria pinnatifida is capable of functioning as a releasing agent after administration to a subject.

In some embodiments, the subject consumes and digests whole Lycium barbarum berries. The berries may be fresh, frozen, freeze-dried, dehydrated, or preserved in some other manner. Therefore, Lycium barbarum, as described herein, encompasses both whole berry, or parts of the Lycium barbarum plant. In one embodiment, the mobilization agent is an extract of Lycium barbarum, or an isolated component or compound extracted from Lycium barbarum, such as a compound found in a polysaccharide-rich fraction of Lycium barbarum extract. Lycium barbarum can be provided alone as an isolated or purified substance, or may be part of a composition including a pharmaceutically acceptable carrier. In one embodiment, Lycium barbarum is capable of functioning as a migration agent. In one embodiment, Lycium barbarum is capable of functioning as a releasing agent.

In various embodiments, the cosmetic composition including one or more stem cell mobilization agents is selected from the group of: Polygonum multiflorum, blue-green algae, fucoidan, and Lycium barbarum. In different embodiments, the composition includes 50-750 mg each for the one or more stem cell mobilization agents in a capsule or pill. This includes, for example, about 50-100 mg, 100-150 mg, 150-200 mg, 200-250 mg, 250-300 mg, 350-400 mg, 400-450 mg, 450-500 mg, 500-550 mg, 550-600 mg, 600-650 mg, 650-700 mg, 700-750. In other embodiments, this includes a composition with less than 300 mg of fucoidan and 50-750 mg each for the one or more stem cell mobilization agents selected from the group of: Polygonum multiflorum, blue-green algae, fucoidan, and Lycium barbarum. In various embodiments, the composition is for use in treating graying hair in a subject. In various embodiments, the composition is for use in modulating melanocytes in the scalp in a subject.

Also described herein is a method for improving the cosmetic appearance of hair in a subject including, providing a quantity of a composition including a stem cell mobilization agent, and a cosmetically acceptable carrier, and administering a cosmetically effective amount of the composition to the subject. In various embodiments, the mobilization agent is a composition including one or more of the following components selected from the group of: Aloe or extracts thereof, Polygonum multiflorum or extracts thereof, Lycium barbarum or extracts thereof, colostrum or extracts thereof, spirulina or extracts thereof, fucoidan, Hericium erinaceus or extracts thereof, Ganoderma Lucidum or extracts thereof, and/or Cordyceps Sinensis or extracts thereof. In various embodiments, the mobilization agent is a composition comprising one or more of the following components selected from the group of: Aloe or extracts thereof, Polygonum multiflorum or extracts thereof, Lycium barbarum or extracts thereof, colostrum or extracts thereof, spirulina or extracts thereof, fucoidan, Hericium erinaceus or extracts thereof, Ganoderma Lucidum or extracts thereof, and/or Cordyceps Sinensis or extracts thereof. In various embodiments, the stem cell mobilization agent includes Polygonum multiflorum, blue-green algae, fucoidan, Lycium barbarum, and combinations thereof. In various embodiments the blue-green algae is a gram-negative photosynthetic bacteria belonging to the division Cyanophyta. In various embodiments, the blue-green algae that is a gram-negative photosynthetic bacteria belonging to the division Cyanophyta includes Spirulina species and Aphanizomenon species. In other embodiments, fucoidan includes Undaria species. In various embodiments, administering a cosmetically effective amount of the composition by a route of delivery, including, for example, non-invasive peroral (through the mouth), topical (skin), transmucosal (nasal, buccal/sublingual, vaginal, ocular and rectal) and inhalation routes, as well as parenteral routes, and other methods know in the art. In certain embodiments, improving the cosmetic appearance of hair in a subject includes reducing, stopping and/or reversing the graying of hair. In other embodiments, improving the cosmetic appearance relates to enhanced migration of melanocyte stem cells into hair follicle locations, increased melanin production, increased melanin content in keratinocytes and/or melanocytes and/or increases populations of keratinocytes and/or melanocytes in the hair scalp.

In various embodiments, improving the cosmetic appearance of hair color, may be quantitatively, or qualitatively measured. For example, a quantitative measure can include colorimetric assays measuring the density of white hair. In other embodiments, qualitative measurements can include improved aesthetic satisfaction to the subject.

Also described herein is a method for treating hair graying in a subject including, providing a quantity of a composition including a stem cell mobilization agent, and a therapeutically acceptable carrier, and administering a therapeutically effective amount of the composition to the subject. In various embodiments, the mobilization agent is a composition including one or more of the following components selected from the group of: Aloe or extracts thereof, Polygonum multiflorum or extracts thereof, Lycium barbarum or extracts thereof, colostrum or extracts thereof, spirulina or extracts thereof, fucoidan, Hericium erinaceus or extracts thereof, Ganoderma Lucidum or extracts thereof, and/or Cordyceps Sinensis or extracts thereof. In various embodiments, the stem cell mobilization agent includes Polygonum multiflorum, blue-green algae, fucoidan, Lycium barbarum, and combinations thereof. In various embodiments the blue-green algae is a gram-negative photosynthetic bacteria belonging to the division Cyanophyta. In various embodiments, the blue-green algae that is a gram-negative photosynthetic bacteria belonging to the division Cyanophyta includes Spirulina species and Aphanizomenon species. In other embodiments, fucoidan includes Undaria species. In other embodiments, treating hair graying relates to enhanced migration of melanocyte stem cells into hair follicle locations, increased melanin production, increased melanin content in keratinocytes and/or melanocytes and/or increases populations of keratinocytes and/or melanocytes in the hair scalp.

In various embodiments, the cosmetic composition including one or more stem cell mobilization agents is selected from the group of: Polygonum multiflorum, blue-green algae, fucoidan, and Lycium barbarum. In different embodiments, the composition includes 50-750 mg each for the one or more stem cell mobilization agents in a capsule or pill. This includes, for example, about 50-100 mg, 100-150 mg, 150-200 mg, 200-250 mg, 250-300 mg, 350-400 mg, 400-450 mg, 450-500 mg, 500-550 mg, 550-600 mg, 600-650 mg, 650-700 mg, 700-750. In other embodiments, this includes a composition with less than 300 mg of fucoidan and 50-750 mg each for the one or more stem cell mobilization agents selected from the group of: Polygonum multiflorum, blue-green algae, fucoidan, and Lycium barbarum. In various embodiments, the composition is for use in treating graying hair in a subject. In various embodiments, the composition is for use in modulating melanocytes in the scalp in a subject.

Further described herein is method of inducing an increase in melanin production in the hair scalp of a subject by providing a quantity of a composition including a stem cell mobilization agent, and a cosmetically and/or therapeutically acceptable carrier, and administering a cosmetically and/or therapeutically effective amount of the composition to the subject. In certain embodiments, the melanin is eumelanin. In other embodiments, the melanin is pheomelanin. In certain embodiments, the increase in melanin production relates to increased melanin content in keratinocytes and/or melanocytes. In other embodiments, the increase in melanin production in the hair scalp relates to an increase in the population of keratinocytes and/or melanocytes.

Further described herein is method of inducing an increase in the population of melanocytes in the hair scalp of a subject, following administration of a stem cell mobilization agent. In one embodiment, increased population of melanocytes in the hair scalp of a subject may be measured by assaying the number of melanocytes in the hair scalp of a subject. In one embodiment, providing stem cell mobilization agent to a subject will increase population of melanocytes in the hair scalp of a subject within a certain time period, such as after 1, 2, 3, 4, 5, 6, 7, 8, 9, 10, 11, 12 or more weeks following administration of the stem cell mobilization agents. In other embodiments, increased population of melanocytes in the hair scalp of a subject will be present after a certain time period, such as after 3, 4, 5, 6, 7, 8, 9, 10, 11, 12 or more months following administration of the stem cell mobilization agent.

Further described herein is method of inducing an increase in the population of circulating stem cells, such as CD34+ stem cells following administration of a stem cell mobilization agent. In one embodiment, the stem cells are hematopoietic stem cells (HSGs). In another embodiment, the stem cells are bone marrow-derived stem cells. In one embodiment, enhancement of stem cell trafficking may be measured by assaying the response of stem cells to a particular dose of stem cell mobilization agent. In one embodiment, providing stem cell mobilization agent to a subject will enhance release of that subject's stem cells within a certain time period, such as less than 12 days, less than 6 days, less than 3 days, less than 2, or less than 1 days. In an alternative embodiment, the time period is less than 12 hours, 6 hours, less than about 4 hours, less than about 2 hours, or less than about 1 hour following administration.

### EXAMPLES

The following examples are provided to better illustrate the claimed invention and are not to be interpreted as limiting the scope of the subject matter. To the extent that specific materials are mentioned, it is merely for purposes of illustration and is not intended to limit the invention. One skilled in the art may develop equivalent means, compositions or reactants without the exercise of inventive capacity and without departing from the scope of the present invention.

### Example 1

### Study Design

Six healthy male Caucasian subjects (ages 46-61) exhibiting significant hair graying on both sides and the back of the head were selected. Criteria for exclusion included allergies, history of hypersensitivity to medications, and being currently under a doctor's care. Each subject came for assessment prior to consuming the stem cell mobilization agent composition, Stemenhance, including Aphanizomenon flus aquae, and then once a month for 6 months while consuming two capsules of stem cell mobilization agent three times a day. Subjects were instructed not to change their hairstyle, grooming products or haircut schedule for the duration of the study. They were also instructed to report any adverse reactions. During each visit, photo-colorimetric assessment was performed to determine white density.

The technicians involved in this study were all “Color Certified” annually by a Board Certified Ophthalmologist using the Farnsworth-Munsell 100 Hue Test. Density of white in the hair was quantified using the Minolta CR-200 Chromameter interfaced with a DP-100 Color Computer System (“Minolta CR-200”), with a focus on the quantification of darkening of the hair color, as perceived by the human eye. The instrument detects subtle changes in color by a three dimensional profile of hue, value and chroma. These characteristics are then translated into color coordinates (a*, b*, and L*) whose spacing is considered to correlate with the color changes perceived by the human eye. An increase in the L* coordinate indicates lightening of the color. Respectively, any diminution of the L* coordinate is indicative of the darkening of color. Hair color was determined in 3 locations: back, right and left sides. At each location, color was determined at 6 positions: the back, upper left corner, upper right corner, lower left corner, lower right corner, and center. For each subject and time, the values used for a*, b* and L* were the average of the 6 positions. Data are expressed as mean±SD. Systat 12 (Systat, Inc., Chicago, Ill.) was used to analyze changes in L* over time, using repeated measures analysis of variance and orthogonal polynomial regression, in order to determine trends. Statistical significance was declared at p≦0.05.

### Example 2

### Changes in Density of White Hair

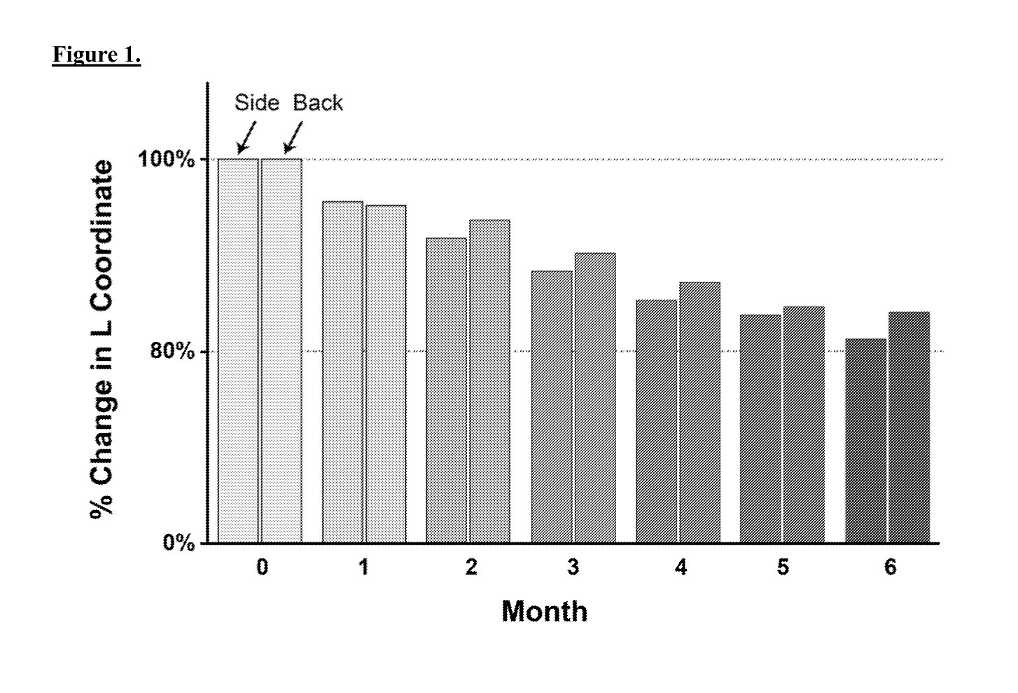

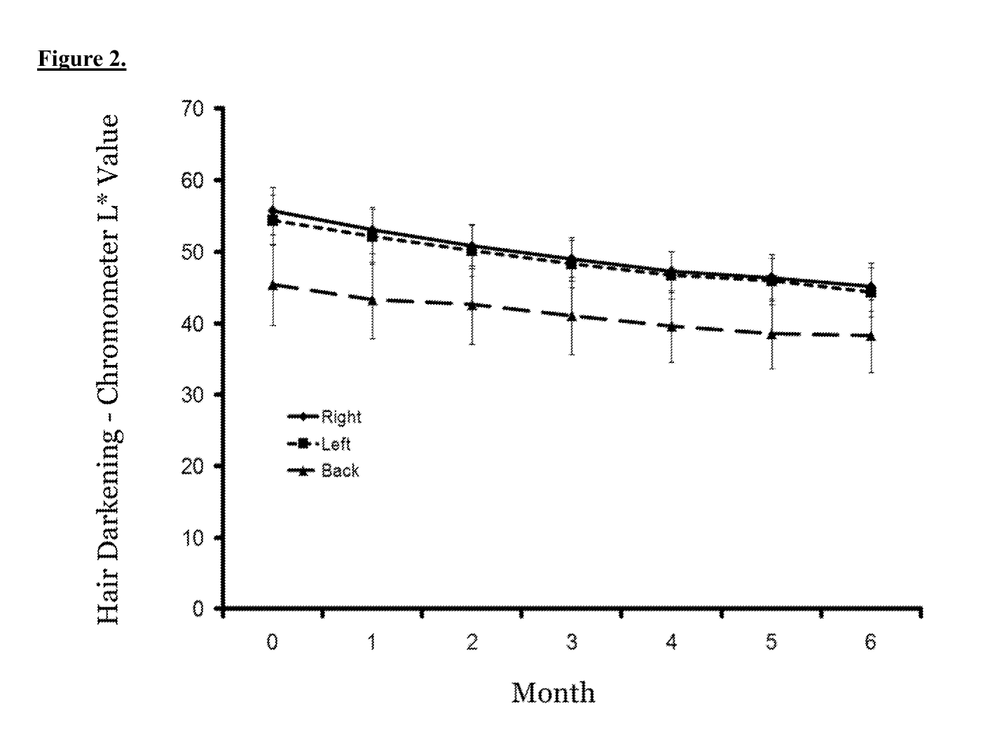

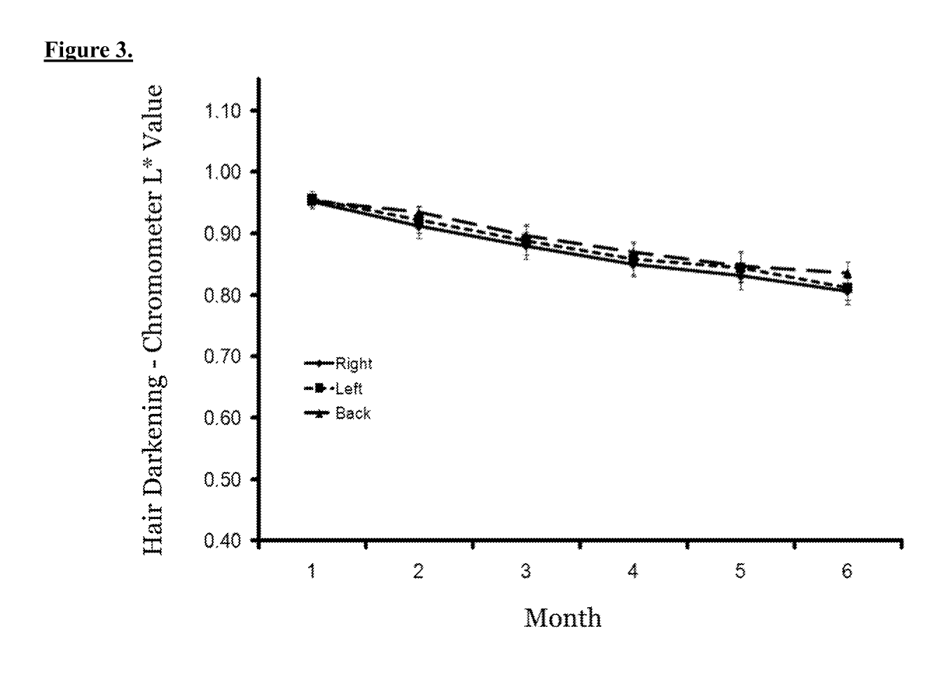

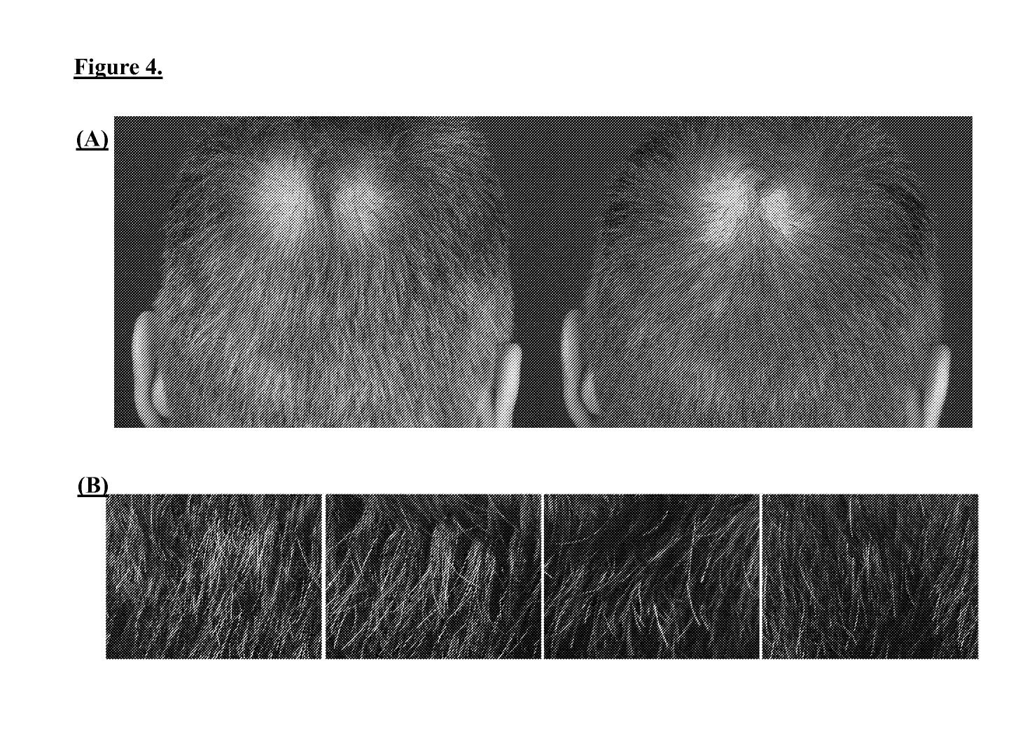

Different measures can describe for the appearance of colors, including three types of objective measures (psychophysical terms, psychometric terms, and psychoquantitative terms) and one type of subjective measure (psychoquantitative terms) can describe for the appearance of colors. L* represents psychometric lightness, and a* and b* represent psychometric chromaticness. As expected, there was no significant trend (or other relationship) over time associated with either a* or b*. However, the expected reduction in the density of white, measured as a diminution of the L* coordinate, using a Minolta CR-200, was seen in all subjects on both sides of the head and in the back (FIG. 1). The diminution of the L* coordinate was linear over time between the initial (0 months) and final (6 months) assessments. The intercept for the back differed from those for the right and left sides (p=0.022), indicating more graying on the sides, but the negative slopes for the right and left sides and the back were similar (p=0.099) (FIG. 2). However, when L* at each time is expressed as its ratio to the pretreatment (month 0) value, the three lines are coincident with a negative linear slope, showing the similar rate of progression of the effect. This diminution in L* directly corresponds to darkening of hair color (FIG. 3). The increases in hair color were more pronounced on the sides of the head (−19.1±4.9%) than in the back (−16.4±4.4%), but the difference between both areas was not significant. Therefore, the data obtained from the sides and back of the head was pooled and the average reduction in white density in these areas gradually reached −18.4±4.6% (p<0.0001) after 6 months of consumption, ranging between −11% and −28.6%. In at least three subjects, empirical observations also suggested a thickening of the hair as well as new growth, as seen in FIG. 4, although this was not quantified.

### Example 3

### Release Agent

### Polygonum multiflorum (He Shou Wu)

When their effect was tested on stem cells, Polygonum multiflorum was documented to support the release of CD34+ stem cells from the bone marrow. In a described study, one can test the efficacy of Polygonum multiflorum compared to placebo, by for example, obtaining peripheral venous blood samples from healthy human volunteers between 20 and 45 years of age, before and after administering an oral composition of Polygonum multiflorum compared or placebo (e.g., potato flakes). In some instances, crushed Polygonum multiflorum root may be diluted in water in a 12:1 extract as a simple preparation. For example, a baseline blood sample can be drawn, and immediately after drawing the baseline sample, the consumable can be provided, followed by further blood sample draws 60 and 120 min after ingestion of the consumable. At each time point, 5 ml of blood was drawn into heparin, and 2 ml blood was drawn into EDTA. The blood vials were placed on a rocking plate until use. Red blood cells in whole blood samples obtained from each volunteer were lysed using FACS lysing solution (Beckton Dickenson, San Jose, Calif.). The remaining cells were washed and stained with monoclonal antibody HPCA-2 conjugated with fluorescein isothiocyanate. Samples were fixed in 1% formalin and analyzed by flow cytometry using a FacsCalibur flow cytometer (Becton Dickenson, San Jose, Calif.) and CellQuest software (Becton Dickenson, San Jose, Calif.).

Using these described methods, consumption of 1 gram of an extract of Polygonum multiflorum triggered a transient increase (18%) in the number of circulating stem cells, thereby demonstrating its potential to function as a mobilization agent, specifically related to release of stem cells into the peripheral blood.

### Example 4

### Migration Agent

### Lycinium barbarum (Gou Qi Zi)

Consumption of Lycium barbarum, or compounds thereof, enhances recruitment and migration of CD34+ stem cells. Using the described methods, with either consumption of 5 grams of dried Lycium barbarum or 1 gram of polysaccharide extracted from Lycium barbarum,

Briefly, extracts may be polysaccharides from Lycium barbarum as prepared by taking the dried fruit samples (100 g) and grinding to fine powder and put in 1.5 l of boiling water and decocted for 2 h by a traditional method for Chinese medicinal herbs. The decoction was left to cool at room temperature, filtered and then freeze-dried to obtain crude polysaccharides. Dried crude polysaccharides are refluxed three times to remove lipids with 150 ml of chloroform:methanol solvent (2:1) (v/v). After filtering the residues are air-dried. The result product is extracted three times in 300 ml of hot water (90° C.) and then filtered. The combined filtrate was precipitated using 150 ml of 95% ethanol, 100% ethanol and acetone, respectively. After filtering and centrifuging, the precipitate was collected and vacuum-dried, giving desired polysaccharides (13 g). Using this technique, the content of the polysaccharides in the extract may reach 97.54%.

When administered, it was observed that consumption of Lycium barbarum triggered a strong transient decrease in circulating stem cells. The peak decrease in circulating CD34+ cells was observed at about 1-2 hours after consumption. At this time point, the number of circulating CD34+ cells was decreased by 24% below the control value, and 4 hours after Lycium barbarum ingestion, the circulating CD34+ cells had returned to the baseline value. Importantly, the decrease in the number of circulating stem cells is accompanied by an increase in the expression of CXCR4 on the membrane of circulating stem cells. Therefore, Lycium barbarum (or a biological component of Lycium barbarum) can enhance the migration of endogenous stem cells (e.g. CD34+ cells) from the circulation to tissues.

### Example 5

### Discussion

Remarkably, two of the rare plants known to support the Jing were also documented by modern scientific techniques to support stem cell function in the body in a manner consistent with traditional viewpoints. Importantly, their mechanisms as Polygonum multiflorum supports the release of stem cells from the bone marrow, while the Lycium barbarum supports their migration into tissues. Together, the general effect remains the support of the natural role of stem cells in the body. It is interesting to note that Polygonum multiflorum is rarely used alone when the intent is to support the Jing or the Kidney or to build the Blood, instead it is blended with plants that can “circulate” the Jing and Lycium barbarum is commonly used for that purpose. The results described herein provide a clear effect of promoting stem cell “circulation” in the blood

### Example 6

### Combinations of Mobilization Agents for Use in Hair Graying

Given the demonstrated capacity of mobilization agents, such as Stemenhance as reducing, stopping, and reversing of hair graying, other mobilization agents such as Polygonum multiflorum, fucoidan, and Lycium barbarum are likely to support similar results, given their shared mechanisms

In this aspect, subjects can consum the stem cell mobilization agent composition, including any of the above mobilization agents, according to the described dosages and for periods of up to 6 months. For example, consuming two capsules of stem cell mobilization agent three times a day is an exemplary dosing regimen.

Using the described measurement techniques, it is expected that a diminution in L*, corresponding to darkening of hair color will be observed.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the sources of stem cell mobilization agents, the methods of preparing, isolating, or purifying stem cell mobilization agents, analogs and derivatives thereof, methods of treating various disease and/or conditions using stem cell mobilization agents, analogs and derivatives thereof, techniques and composition and use of solutions used therein, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method for improving the cosmetic appearance of hair in a subject, comprising:
providing a quantity of composition comprising one or more stem cell mobilization agents selected from the group consisting of: Polygonum multiflorum, blue-green algae, fucoidan, and Lycium barbarum, and a cosmetically acceptable carrier; and
administering a cosmetically effective amount of the composition to the subject, wherein administration of the composition improves the cosmetic appearance of hair in a subject.

2. The method of claim 1, wherein the stem cell is a bone marrow-derived stem cell (BMSC).

3. The method of claim 1, wherein the stem cell is a hematopoietic stem cell (HSC).

4. The method of claim 1, wherein administering the quantity comprises oral administration.

5. The method of claim 4, wherein oral administration comprises use of a capsule or a pill.

6. The method of claim 5, wherein the capsule or a pill comprises 50-750 mg each for the one or more stem cell mobilization agents.

7. The method of claim 6, wherein the capsule or a pill comprises less than 300 mg of fucoidan and 50-750 mg each for the one or more stem cell mobilization agents selected from the group consisting of: Polygonum multiflorum, blue-green algae, fucoidan, and Lycium barbarum.

8-13. (canceled)

14. A method of modulating melanocytes in the hair scalp of a subject, comprising:
providing a quantity of composition comprising one or more stem cell mobilization agents selected from the group consisting of: Polygonum multiflorum, blue-green algae, fucoidan, and Lycium barbarum; and
administering the composition to the subject, wherein administration of the composition modulating melanocytes in the hair scape of the subject.

15. The method of claim 14, wherein modulating melanocytes in the hair scalp of the subject comprises increasing the population of melanocytes in the hair scalp and/or a hair follicle niche.

16. The method of claim 14, wherein modulating melanocytes in the hair scalp of the subject comprises increasing the production of melanin by melanocytes.

17. The method of claim 14, wherein modulating melanocytes in the hair scalp of the subject comprises increasing level of melanin keratinocytes.

18. The method of claim 14, wherein the composition comprises 50-750 mg each for the one or more stem cell mobilization agents in a single dose.

19. The method of claim 14, wherein the composition comprises less than 300 mg of fucoidan and 50-750 mg each for the one or more stem cell mobilization agents selected from the group consisting of: Polygonum multiflorum, blue-green algae, fucoidan, and Lycium barbarum.

20. A method for treating graying hair in a subject, comprising:
providing a quantity of composition comprising one or more stem cell mobilization agents selected from the group consisting of: Polygonum multiflorum, blue-green algae, fucoidan, and Lycium barbarum, and a cosmetically acceptable carrier; and
administering a therapeutically effective amount of the composition to the subject, wherein administration of the composition treats hair graying in the subject.

21. The method of claim 20, wherein the composition comprises 50-750 mg each for the one or more stem cell mobilization agents.

22. The method of claim 20, wherein the composition comprises less than 300 mg of fucoidan and 50-750 mg each for the one or more stem cell mobilization agents selected from the group consisting of: Polygonum multiflorum, blue-green algae, fucoidan, and Lycium barbarum.

24. The method of claim 20, wherein the composition modulates melanocytes in the scalp in a subject.

25. The method of claim 20, wherein modulating melanocytes in the hair scalp of the subject comprises increasing the production of melanin by melanocytes.
